(19) United States
(12) Patent Application Publication
(10) Pub. No.: US 2009/0175934 A1
(43) Pub. Date: Jul. 9, 2009

(54) Extended Release Pharmaceutical Formulation of Venlafaxine and Method of Manufacturing the Same

(75) Inventors: Nagesh Nagaraju, Karnataka (IN); Manish Dhall, Haryana (IN); Gour Mukherji, Haryana (IN); Satya Sankar Sahoo, West Bengal (IN)

Correspondence Address: COHEN, PONTANI, LIEBERMAN & PAVANE LLP, 551 FIFTH AVENUE, SUITE 1210, NEW YORK, NY, 10176, US

(73) Assignee: Jubilant Organosys Ltd., Noida (IN)

(21) Appl. No.: 12/224,910

(22) PCT Filed: Feb. 21, 2007
(86) PCT No.: PCT/IN2007/000069

§ 371 Date: Sep. 8, 2008

(30) Foreign Application Priority Data
Mar. 8, 2006 (IN) 595/DEL/2006

(51) Int. Cl. A61K 9/48; A61K 31/137; A61K 9/32; A61P 25/22; A61P 25/24

(52) U.S. Cl. 424/456; 514/654; 424/482; 424/451; 427/2.14

(57) ABSTRACT

Disclosed herein is an extended release pharmaceutical formulation suitable for once daily administration, comprising a highly water soluble core consisting essentially of about 30 to about 40% by weight of venlafaxine hydrochloride, about 50 to about 80% by weight of water soluble diluent and about 2 to about 10% of water soluble binder and a coating layer having an effective combination of rate controlling polymers comprising water-soluble polymer and water insoluble, water permeable polymer.

### FIELD OF THE INVENTION

This invention in general, relates to a pharmaceutical formulation for anti-depressant drug. More particularly, the present invention provides an extended release pharmaceutical formulation comprising a therapeutically effective amount of Venlafaxine or its pharmaceutically acceptable salts and process for preparing the same.

### BACKGROUND OF THE INVENTION

Venlafaxine, 1-[2-dimethylamino)-1-(4-methoxyphenyl)ethyl]cyclohexanol, is an important drug in the neuropharmacological arsenal used for treatment of depression. Venlafaxine and the acid addition salts thereof are disclosed in U.S. Pat. No. 4,535,186. Venlafaxine hydrochloride is presently administered to adults in compressed tablet form in doses ranging from 75 to 350 mg/day, in divided doses two or three times a day. In therapeutic dosing with Venlafaxine hydrochloride tablets, rapid dissolution results in a rapid increase in blood plasma levels of the active compound shortly after administration followed by a decrease in blood plasma levels over several hours as the active compound is eliminated or metabolized, until sub-therapeutic plasma levels are approached after about twelve hours following administration, thus requiring additional dosing with the drug. With the plural daily dosing regimen, the most common side effect is nausea, experienced by about forty five percent of patients under treatment with Venlafaxine hydrochloride. Vomiting also occurs in about seventeen percent of the patients.

U.S. Pat. No. 6,696,496 discloses the low soluble salts of Venlafaxine containing once daily dosage form including hydrogel based formulations. The said dosage form comprises of tablets or dosage forms consisting of pellets.

U.S. Pat. No. 6,274,171 assigned to American Home Products Corporation teaches an extended release composition of Venlafaxine hydrochloride in the form of spheroids.

The spheroids are made up of Venlafaxine hydrochloride, microcrystalline cellulose, and hydroxypropyl methylcellulose. The spheroids are coated with a film coating composition comprised of ethyl cellulose and hydroxypropyl methylcellulose, which provides the extended release profile. This composition requires high cost excipients and equipment leading to high cost of the product. Besides, the method of production is tedious, time consuming and skilled labor intensive. It further discloses numerous attempts made to produce extended release tablets by hydrogel technology proved to be fruitless because the compressed tablets were either physically unstable (poor compressibility or capping problems) or dissolved too rapidly in dissolution studies.

U.S. Pat. No. 6,717,015 assigned to Synthon BV, discloses an extended release tablet of Venlafaxine besylate monohydrate, hydroxypropyl methylcellulose and magnesium stearate, as prepared by direct compression method. However, such a tablet has questionable release profile to suit the need for once a day dosage form.

U.S. Pat. No. 4,138,475 discloses a sustained release pharmaceutical composition of a hard gelatin capsule filled with film-coated spheroids comprised of Propranolol in admixture with microcrystalline cellulose wherein the film coating is composed of ethyl cellulose, optionally, with hydroxypropylmethylcellulose and/or a plasticizer.

U.S. Pat. No. 6,703,044 discloses an extended release once daily formulation of Venlafaxine or its pharmaceutically acceptable salts in minitablets encapsulated in capsules. The prior art discloses a composition efficient to provide a pharmaceutically acceptable composition including microcrystalline cellulose as a hardening agent having desired release of Venlafaxine after 3 hours of intake so as to provide a better control of blood plasma levels than conventional tablets.

US Application No. 2004/0131677 describes a programmed release composition including 10% to 80% Venlafaxine HCl by weight. Micronized Venlafaxine HCl is deposited on an inert core using a PVP alcoholic solution in a coating pan to obtain microgranules. The microgranules are coated with talc using the PVP solution and further coated with a plasticized ethylcellulose solution. The yield is not more than 92% by weight. This process requires periodically powdering the product with talc to diminish the static load, thereby interrupting the continuity of process and making it unsuitable for industrial application. The obtained microgranules are not of adequate strength as the mechanical conditions in the fluid bed processor during the coating process causes rupturing of some of the microgranules, which further reduces the yield of the process.

PCT Publication WO 03/041692 is directed to extended release compositions that include Venlafaxine HCl in a concentration of 30% to 60% by weight. The Venlafaxine HCl is coated with a binder having a concentration of 0.5% to 10% by weight on an inert core. This coated core is then coated with an isolating layer and further coated with polymer layer. The process utilizes water, ethanol, or a combination thereof, as a solvent mixture for spraying the Venlafaxine HCl. The process of utilizing water for spraying Venlafaxine HCl as described results in the settling of product mass in a product container, thereby interrupting the continuity of the process. The process of utilizing ethanol as described therein is not sufficient in dissolving Venlafaxine HCl and the Venlafaxine HCl suspension in ethanol, when sprayed on an inert core utilizing PVP as a binder in a concentration of 0.5% to 10% by weight, will result in improper fluidization or changes in fluidization patterns during the process. This leads to inefficient loading of Venlafaxine HCl on inert seeds and results in drug loss and low batch yield, which is generally not more than 95% by weight.

In accordance with aforementioned prior arts regarding the extended release formulation of Venlafaxine either requires high cost excipients or equipments leading to high cost of the product or both. Besides the method of production, it is tedious, time consuming and requires intensive skilled labor.

Therefore, it necessitates need for an extended release formulation of Venlafaxine or its pharmaceutically acceptable salts using low cost excipients, simple and conventional equipments and less skilled labor-intensive method and yet providing the desired drug release profile.

### SUMMARY OF THE INVENTION

It is a principal object of the present invention to provide an extended release pharmaceutical formulation comprising Venlafaxine or its pharmaceutical acceptable salts that exhibits reduced toxicity with desired extended release of the drug.

It is another object of the present invention to provide a cost efficient method to manufacture said extended release pharmaceutical formulation comprising Venlafaxine or its pharmaceutical acceptable salts using low cost excipients and equipments.

Further object of the present invention is to provide an extended release pharmaceutical formulation comprising Venlafaxine or its pharmaceutical acceptable salts that avoids the incidence of drug leaching or dose dumping of said Venlafaxine or its salts from said formulation.

Yet another object of the present invention is to provide an extended release formulation comprising Venlafaxine or its pharmaceutical acceptable salts, which provides a therapeutic blood serum level over a 24 hour period in a single dose thereby reducing the level of nausea and incidence of emesis that cause during the administration of multiple daily dosing.

The above and other objects of the present invention are further described in accordance with following embodiments, however it is not limited to the scope of the present invention.

In accordance with one preferred embodiment of the present invention, there is provided an extended release formulation of Venlafaxine or its pharmaceutically acceptable salts, wherein said formulation comprising a highly water soluble core having said Venlafaxine or its pharmaceutically acceptable salts with conventional excipients and a coating layer having an effective combination of rate controlling polymers.

In accordance with another preferred embodiment of the present invention, there is provided an extended release formulation of Venlafaxine or its pharmaceutically acceptable salts, wherein said a highly water soluble core of formulation consisting essentially of about 30 to about 40% by weight of Venlafaxine or its pharmaceutically acceptable salts, high amount of water soluble diluent preferably from about 50 to about 80% by weight and a water soluble binder preferably from about 2 to about 10% by weight. All weight percentages as mentioned herein are based on the total weight of the core or uncoated tablet.

The highly water soluble core includes Venlafaxine hydrochloride, and conventional excipients, notably a water soluble diluent and a water soluble binder, and optionally other excipients and wherein said core is essentially free of rate controlling polymer.

In accordance with the preferred embodiment, said core is coated with a coating layer employing rate controlling polymers, which comprises of an effective combination of water insoluble, water permeable polymer and water-soluble polymer, preferably about 60 to about 80% of water insoluble, water permeable polymer and about 20 to about 40% of water soluble polymer

In accordance with yet another preferred embodiment of the present invention, there is provided an extended release formulation of Venlafaxine or its pharmaceutically acceptable salts, wherein said formulation is once daily dosage form in the form of tablets of uniform size measuring about 1 mm to about 12 mm. Further said tablets may be encapsulated in a hard gelatin capsules or hydroxyl propyl methyl cellulose [HPMC] capsules.

In accordance with still another preferred embodiment of the present invention, there is provided an extended release formulation of Venlafaxine or its pharmaceutically acceptable salts, wherein said formulation is prepared by a method comprising the steps of: (a) mixing Venlafaxine or its pharmaceutically acceptable salt and diluent, (b) granulating the resultant mixture with aqueous/non-aqueous solution of water soluble binder and drying the so formed granules, (c) lubricating the dried granules and compressing them into tablets, (d) coating the tablets with dispersion or solution or suspension of polymers comprising water insoluble, water permeable polymer and water soluble polymer in a suitable coating apparatus and (e) encapsulating the tablets into hard gelatin capsule or HPMC capsule.

The formulation disclosed in accordance with the present invention is effective for the treatment of major depressive disorder, social anxiety disorder, generalized anxiety disorder and panic disorder.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The extended release formulation of Venlafaxine in accordance with the present invention, consists essentially of Venlafaxine or its pharmaceutically acceptable salts, water soluble diluents and water soluble binders, wherein said formulation is in the form of tablets. The formulation may further comprise other pharmaceutical excipients, suitable for the preparation of tablet formulation.

The tablet according to the present invention provides desired drug release profile to suit the once-a-day dosing of Venlafaxine or its pharmaceutically acceptable salts. The Venlafaxine may be used in any polymorphic form e.g. Polymorph I and Polymorph II as known in the art.

The Venlafaxine or its pharmaceutically acceptable salt used herein, is in an amount from about 25 to about 50% by weight, preferably from about 30 to about 40% by weight in said formulation. The preferred salt is Hydrochloride salt of Venlafaxine.

Suitable water soluble diluents used in the formulation include, but are not limited to, lactose, sorbitol, mannitol, sucrose, dextrose, dextrates or dextrin or any combination thereof. The preferred diluent is lactose. Further the diluent is preferably used in an amount from about 50 to about 80% by weight.

Suitable binders used in the formulation are essentially water soluble and selected from the group comprising polyvinylpyrrolidone, modified starch, hydroxypropylcellulose, hydroxymethyl cellulose, and hydroxyethyl cellulose or hydroxypropyl methylcellulose or a combination thereof. The preferred binder is polyvinylpyrrolidone. Further the binder is preferably used in an amount from about 2 to about 10% by weight.

The extended release formulation of Venlafaxine in accordance with the present invention, is in the form of tablets, wherein said tablets are cylindrical with convex upper and lower side and with a diameter and height preferably unequal and independent of each other. The size of the tablet may vary from about 1 mm to about 12 mm, preferably from about 2 to about 8 mm. The weight of the tablets depends on the size of the tablets.

The said tablets according to the invention may be filled into suitable size capsule or may be packed in the high-density polyethylene (HDPE) bottle or any other suitable packing. The number of tablets filled into capsule depends on the size of the tablet, as a general guide the number of the tablets filled into capsule decreases as the size of the tablet increases. Therapeutically effective amount of Venlafaxine or its pharmaceutically acceptable salt remains same in each of the capsule, which forms single dosage unit.

The formulation of the present invention in the form of tablets is coated with rate controlling polymers. The rate controlling polymers comprise water insoluble, water permeable polymers and water-soluble polymers.

Rate controlling polymers as used herein refer to the polymers that retard the release of the drug from the given dosage form.

Suitable water insoluble, water permeable polymers include but are not limited to cellulose ether, such as low viscosity ethylcellulose, a cellulose ester, such as cellulose acetate, polyvinylalcohol, copolymers of acrylate and methacrylates with quarternary ammonium group such as, EUDRAGIT® RL30 D and EUDRAGIT® RS 30 D (available from Rohm Pharma). EUDRAGIT® RL30 D is chemically known as Poly (ethyl acrylate, methyl methacrylate, trimethylammonioethyl methacrylate chloride) 1:2:0.2. Similarly EUDRAGIT® RS 30 D is chemically known as Poly (ethyl acrylate, methyl methacrylate, trimethylammonioethyl methacrylate chloride) 1:2:0.1. The preferred water insoluble and water permeable polymer is low viscosity (10-50 cps) ethylcellulose (available from Dow Chemical and Hercules under the trade name ETHOCEL® and AQUALON® respectively). Viscosity determination of ethylcellulose is done on the basis of 5% solution in 80:20 mixture of toluene and ethanol.

Suitable water-soluble polymers of the present invention include but are not limited to polyvinylpyrrolidone, Poly (ethylene oxide), hydroxymethyl cellulose and low viscosity (5-15 cps) hydroxypropyl methylcellulose. The preferred water-soluble polymer is low viscosity (5-15 cps) hydroxypropyl methylcellulose. Hydroxypropyl methylcellulose is commercially available from Shin-Estu Co., Japan under the brand name PHARMACOAT® 6 cps. The viscosity of the hydroxypropyl methylcellulose is determined on the basis of its 2% aqueous solution.

Suitable water insoluble, water permeable polymer is preferably used in an amount from about 60 to 80% by weight of the coating and water-soluble polymer is preferably used in an amount of from about 20 to about 40% by weight of the coating. The rate controlling polymer coating is used in an amount from about 1 to about 20% by weight of the core. Preferably, rate controlling polymer coating is used in an amount from about 4 to about 15% by weight of the core.

The coating component of the present invention may further contain a plasticizer, colorants and necessary solvents to dissolve the polymers. The examples of these excipients are well known in the pharmaceutical art.

The formulation of the invention in the form of tablets is prepared by the steps comprising (a) mixing venlafaxine or its pharmaceutically acceptable salt and diluent, (b) granulating the resultant mixture with aqueous/non-aqueous solution of binder and drying the so formed granules, (c) lubricating the dried granules and compressing them to tablets, and (d) coating the tablets with dispersion or solution or suspension of polymers comprising water insoluble, water permeable polymer and water-soluble polymer and (e) encapsulating the resultant tablets into suitable size hard gelatin capsule or HPMC capsule using suitable encapsulator equipped with a feeder.

The coating solution is prepared by dispersing water-soluble polymer and water insoluble, water permeable polymer in the isopropyl alcohol with constant stirring, followed by addition of colorant, dispersing in appropriate quantity of water.

Mixing and granulation can be carried out in a suitable apparatus like rapid mixer granulator (RMG) or fluid bed processor. Drying of granules can be carried out in fluid bed processor itself or other drying techniques can be employed such as tray drying, vacuum drying, flash drying etc. Mixing of dried granules with lubricants can be carried out in a suitable mixer like double cone or V-blender or Conta blender. The tableting of lubricated granules can be carried out in a suitable tabletting machine equipped with suitable tooling. Coating of tablets can be carried out in a suitable coating apparatus like coating pan or fluid bed processor. Encapsulation of tablets into hard gelatin capsule or HPMC capsule can be carried out in any suitable encapsulator equipped with a suitable feeder.

The formulation of the invention in the form of tablet filled into capsules is particularly suitable for once-a-day oral dosing of Venlafaxine hydrochloride for the method of treating major depressive disorder, social anxiety disorder, generalized anxiety disorder and panic disorder.

The dissolution profile of the formulation of the invention in the form of tablets is comparable with the dissolution profile of EFFEXOR®XR. EFFEXOR®XR is the commercially available extended release pharmaceutical formulation in the form of spheroids filled into capsules, meant for once-a-day dosing of Venlafaxine hydrochloride.

The following examples further illustrate the present invention. They are, however, not intended to be limiting the scope of the present invention in any way.

### Example 1 to 4

### Venlafaxine Extended Release Formulation

### I) Tablet Composition: (Table 1)

TABLE 1

| | | Amount of Ingredient (in mg)/cap | | | |
|---|---|---|---|---|---|
| Sl. No. | Ingredient | Example 1 | Example 2 | Example 3 | Example 4 |
| 1 | Venlafaxine hydrochloride | 170.0 (150) | 169.7 (150) | 169.7 (150) | 169.7 (150) |
| | Equivalent to Venlafaxine | | | | |
| 2 | Lactose | 270.0 | 277.3 | 277.3 | 277.3 |
| 3 | Polyvinylpyrrolidone (K-30) | 20.0 | 15.0 | 15.0 | 15.0 |
| 4 | Talc | 2.0 | — | — | — |
| 5 | Colloidal silicon dioxide | 2.0 | 2.0 | 2.0 | 2.0 |
| 6 | Magnesium stearate | 2.0 | 5.0 | 5.0 | 5.0 |
| 7 | Purified Water* | q.s | q.s | — | q.s |
| 8 | Isopropyl alcohol | — | — | q.s | — |
| | Total | 466.0 | 469.0 | 469.0 | 469.0 |

### II) Coating Composition (Table 2)

TABLE 2

| | | Amount of Ingredient (in mg)/cap | | | |
|---|---|---|---|---|---|
| SL. No. | Component | Example 1 | Example 2 | Example 3 | Example 4 |
| 1 | Ethyl cellulose N50 | 28.0 | 14.26 | 31.93 | 26.39 |
| 2 | Hydroxypropyl methylcellulose | 5.0 | 2.51 | 10.64 | 17.60 |
| | 6 cps | | | | |
| 3 | Isopropyl Alcohol* | q.s. | q.s | q.s. | q.s |
| 4 | Yellow oxide of iron | 1.0 | 0.10 | 0.10 | 0.10 |
| 5 | Purified water* | q.s. | q.s | q.s | q.s |

*Evaporates during processing

### III) Coating Weight Gain on Tablet (Table 3)

TABLE 3

| Component | Example 1 | Example 2 | Example 3 | Example 4 |
|---|---|---|---|---|
| Coating weight gain (%), | 1.5-2.5 | 2.5-4.0 | 4.0-8.0 | 8.0-18.0 |
| based on the weight of | | | | |
| the uncoated tablet | | | | |

Manufacturing Procedure (tablet): (Examples 1-4)
a) Venlafaxine hydrochloride and lactose were passed through suitable size mesh and mixed.
b) Contents of (a) were granulated with aqueous/non-aqueous solution of polyvinylpyrrolidone (K-30).
c) The wet mass was dried and passed through suitable size mesh.
d) The dried granules of stage (c) were blended with magnesium stearate, colloidal silicon dioxide and optionally with talc in a suitable blender and lubricated granules were compressed into tablets on a compression machine.

### Coating:

e) The tablets obtained in stage (d) were coated with coating solution, prepared by dispersing hydroxypropyl methylcellulose and ethyl cellulose in the isopropyl alcohol with constant stirring, followed by addition of yellow oxide of iron, dispersed in appropriate quantity of water and stirred until uniform dispersion of solution was formed.
f) The coated tablets were filled in suitable size capsules using a suitable encapsulator fitted with a feeder.

### Dissolution Studies

The tablets of the invention were subjected to an in vitro dissolution method to determine the rate at which the drug Venlafaxine hydrochloride was released from the tablets/or tablets filled into capsules. Dissolution method involved USP type I apparatus at 100 rpm wherein water was used as dissolution medium at 37° C. temperature. The dissolution profiles of the formulations of example 1 to 4 were compared with the EFFEXOR®XR and the results are summarized in the table 4 below. EFFEXOR®XR is the commercially available extended release pharmaceutical formulation in the form of spheroids filled into capsules, meant for once-a-day dosing of Venlafaxine hydrochloride.

TABLE 4

| | Dissolution profiles | | | | |
|---|---|---|---|---|---|
| | (Drug released in terms of percentage | | | | |
| | of Venlafaxine hydrochloride) | | | | |
| | Exam- | Exam- | Exam- | Exam- | |
| Time | ple 1 | ple 2 | ple 3 | ple 4 | EFFEXOR ®XR |
| 4 hours | 42 | 32 | 29 | 76 | 41 |
| 8 hours | 61 | 52 | 78 | 92 | 68 |
| 12 hours | 72 | 63 | 94 | 94 | 80 |
| 24 hours | 85 | 77 | 95 | 94 | 91 |

The results in table 4 above clearly show that the dissolution profiles of formulation of the present invention in the form of tablets filled into capsules (Ex.1, 2, 3) are comparable to the dissolution profile of the commercially available EFFEXOR® XR capsules.

While this invention has been described in detail with reference to certain preferred embodiments, it should be appreciated that the present invention is not limited to those precise embodiments rather, in view of the present disclosure, which describes the current best mode for practicing the invention, many modifications and variations would present themselves to those skilled in the art without departing from the scope and spirit of this invention.

1. An extended release pharmaceutical formulation of Venlafaxine or its pharmaceutical acceptable salt, suitable for once daily administration comprising:
a highly water soluble core consisting essentially of about 30 to about 40% by weight of Venlafaxine hydrochloride, about 50 to about 80% by weight of water soluble diluent and about 2 to about 10% of water soluble binder, said core being essentially free of rate controlling polymer, and
a coating layer coated over said core comprising rate-controlling polymers in a combination of about 60 to about 80% of water insoluble-water permeable polymer and about 20 to about 40% of water-soluble polymer.

2. The pharmaceutical formulation according to claim 1, wherein said diluent is selected from the group consisting of lactose, sorbitol, mannitol, sucrose, dextrose, dextrates, dextrin or any combination thereof.

3. The pharmaceutical formulation according to claim 2, wherein said diluent is lactose.

4. The pharmaceutical formulation according to claim 1, wherein said binder is selected from the group consisting of polyvinylpyrrolidone, modified starch, hydroxypropylcellulose, hydroxymethyl cellulose, hydroxyethyl cellulose, and hydroxypropyl methylcellulose or any combination thereof.

5. The pharmaceutical formulation according to claim 4, wherein said binder is polyvinylpyrrolidone.

6. The pharmaceutical formulation according to claim 1, wherein said rate controlling coating is used in an amount from about 2 to 20% by weight of core.

7. The pharmaceutical formulation according to claim 1, wherein said water insoluble, water permeable polymer is selected from the group consisting of ethylcellulose, cellulose acetate or polyvinylalcohol or copolymers of acrylate and methacrylates with quarternary ammonium group.

8. The pharmaceutical formulation according to claim 7, wherein said water insoluble, water permeable polymer is ethylcellulose.

9. The pharmaceutical formulation according to claim 1, wherein said water-soluble polymer is selected from the group consisting of polyvinylpyrrolidone, polyethylene oxide, hydroxymethyl cellulose or hydroxypropyl methylcellulose or any combination thereof.

10. The pharmaceutical formulation according to claim 9, wherein said water-soluble polymer is hydroxypropyl methylcellulose.

11. The pharmaceutical formulation according to claim 1, wherein the formulation is in a tablet form having uniform size measuring from about 1 mm to about 12 mm.

12. The pharmaceutical formulation according to claim 11, wherein said tablets are encapsulated into a capsule.

13. The pharmaceutical formulation according to claim 12, wherein said capsule is a hard gelatin capsule or an HPMC capsule.

14. The pharmaceutical formulation according to claim 1, wherein said formulation is prepared by a process comprising the steps of:
(a) mixing venlafaxine hydrochloride and water soluble diluent,
(b) granulating the resultant mixture with aqueous/non-aqueous solution of binder and drying the so formed granules,
(c) lubricating the dried granules and compressing the resultant granules to tablets,
(d) coating the tablets with dispersion or solution or suspension of polymers comprising water insoluble, water permeable polymer and water soluble polymer using a suitable coating apparatus, and
(e) encapsulating the resultant formulation into a capsule.

15. The pharmaceutical formulation according to claim 14, wherein said capsule is a hard gelatin capsule or an HPMC capsule.

16. The pharmaceutical formulation according to claim 1, wherein the formulation is used for treatment of major depressive disorder, social anxiety disorder or panic disorder.
